(19) United States
(12) Patent Application Publication
(10) Pub. No.: US 2026/0060596 A1
(43) Pub. Date: Mar. 5, 2026

(54) SYSTEMS AND METHODS FOR INTRACRANIAL NEURAL MONITORING AND MODULATION VIA WIRELESS MEANS

(71) Applicant: BIONAUT LABS INC., Los Angeles, CA (US)

(72) Inventors: Michael SHPIGELMACHER, Los Angeles, CA (US); Alex KISELYOV, San Diego, CA (US); William LOUDON, San Clemente, CA (US); Michael KARDOSH, Kiryat Ono (IL); Eran OREN, Tel Aviv (IL)

(73) Assignee: BIONAUT LABS INC., Los Angeles, CA (US)

(21) Appl. No.: 19/311,994

(22) Filed: Aug. 27, 2025

Related U.S. Application Data
(60) Provisional application No. 63/690,962, filed on Sep. 5, 2024.

(51) Int. Cl. A61B 5/37; A61B 5/293; A61B 5/377; H04B 10/40; H04B 11/00; H04B 13/00

(52) U.S. Cl. CPC A61B 5/37; A61B 5/293; A61B 5/377; H04B 10/40; H04B 11/00; H04B 13/005; A61B 2562/046

(57) ABSTRACT

The invention relates to a system for the wireless monitoring of neural activity via minimally invasive, repositionable means. The use of either optical or direct electrical galvanic communications has the advantage of being safe over a wide range of parameters and allows high rates of reliable analog or digital communications. A small component that is typically driven in an untethered way is integrated with a mechanism that can register electric voltage between at least two points and transmit the resulting recorded signal. A repositionable, small component allows for versatile and safe monitoring of different intracranial sites. A wireless reliable communication method allows reconstructing high-fidelity signals and thus understanding brain activity in places that are otherwise inaccessible.

### CROSS REFERENCE TO RELATED APPLICATIONS

This application claims the benefit of priority of U.S. Provisional Application No. 63/690,962, filed Sep. 5, 2024, hereby incorporated by reference.

### FIELD OF THE INVENTION

The invention herein relates to tools for monitoring and modulating electrical activity in the brain, and more specifically to minimally invasive tools to establish monitoring, communications energy transfer and on-going real-time access to deep brain activity.

### BACKGROUND OF THE INVENTION

The invention herein relates to tools for monitoring and modulating electrical activity. The human nervous system, both central and peripheral, is an electrical and electrochemical machine in its essence. As such, electrical monitoring and modulation of nerve activity holds great promise in many medical aspects. However, limited accessibility to deep brain circuits, and accompanying health risks to invasive procedures relating to the physical access to the brainstem and other parts in the brain, have limited research and treatment capabilities.

A myriad of publications has shown means and methods intended for the collection of viable information regarding brain activity for purposes that range from basic research, diseases diagnostics, treatment of conditions and illnesses, pain management and more. The need for reliable neural activity measurement can be seen for example in [U.S. Pat. No. 7,774,047B2] describing a set of optical sensors in a lattice to perform optical based measurement of blood flow in neurons in a transcranial system (a concept referred to as functional near-IR measurement), which when juxtaposed against heavy machines such as PET, SPECT and fMRI system, that may allow insight into neural activity, but are unlikely to be used over long periods, or allow patients to be monitored in an ambient state. Near infra-red systems are limited by the depth of penetration, thus electric sensing, and specifically Electric Encephalography (EEG) is the dominant option for a variety of medical indications diagnostics and monitoring. To allow ongoing EEG sensing, publications such as [U.S. Pat. Nos. 9,113,801B2], [9,854,985B2], [US20090088608A1], [U.S. Pat. Nos. 8,108,036B2], [8,449,471B2], [10,039,445B1] show a plurality of sensors including a mesh of EEG sensing electrodes connected to via some wireless communications to a controller for the purposes of recording, monitoring or alerting of patients'status. While other publications show the use of similar sensing in conjunction with external triggering to measure evoked potentials such as [JP4833202B2], [U.S. Pat. No. 9,700,228B2]. The wireless connectivity and the variety of sensors shown in said publications and others can be useful for ongoing diagnostics of such conditions as epilepsy, movement disorders and others, but tend to be limited due to the statistical nature of transcranial measurements. While EEG provided information includes data of deep parts of the brain, the attenuation and the statistical nature of measuring large volumes simultaneously, limits the ability to draw conclusions, and gain spatial insight.

To overcome the limitations of cranial EEG, the art has shown internal, intracranial or implanted devices. Publication [U.S. Pat. No. 10,321,866B2] has shown a method to use an implantable device with multiple electrodes to monitor and alert a neurological event such as a seizure. [US20060264774A1] has further shown the use of an implantable device using RFID communications with an external device for neural monitoring, and [U.S. Pat. No. 8,958,868B2] with a general RF interface; [U.S. Pat. No. 9,898,656B2] showed several methods to report seizures from an implantable device via an analysis of multiple points'readouts; [US20090312646A1] suggested internally illuminating a suspected tissue with light via such a tools as an optical fiber to detect a neural event via the scattered light; [U.S. Pat. No. 10,556,132B2] showed an ultrasound communications means of an implantable intracranial sensor; [US20180333587A1], [US20200298005A1] have shown a mechanism for multiple electrodes connected with wires and multiplexers for the same purpose; [WO2022198142A1] suggested a system with optical energy transmission and readout for BCI.

The art has further shown systems of electrical monitoring for purposes of brain-control interfaces (BCI) with invasive or non-invasive electrode arrays and combinations thereof. [U.S. Pat. No. 9,211,078B2] showed an array of electrodes with signals classification;

In some publications, electrodes placed for sensing were shown to be used for modulating neural activity via electrical stimulation. For example, [U.S. Pat. No. 9,409,028B2] has shown a method of placing an elongated microstimulator in an intracranial location, [US20070100398A1] has shown an optical monitoring system connected to a neural stimulator. Specific implementations of nerve stimulation devices were shown with and without sensing mechanisms such as [U.S. Pat. No. 11,389,103B2], [US20120029601A1] showing the stimulation of the Vagus nerve through the neck with and without EEG feedback.

The aforementioned art has taught a wide variety of means and applications for electric and electrochemical interface in the brain. However, some volumes in the deep brain are still well beyond reach for the state-of-the-art, and the inability to move, reposition or perform multiple measurements simultaneous measurement from variable areas, all limit the applicability, safety and efficacy of existing systems.

### BRIEF SUMMARY OF THE INVENTION AND ITS ADVANTAGES

The invention herein is a system for the wireless monitoring of neural activity via minimally invasive, repositionable means. The use of either optical or direct electrical galvanic communications has the advantage of being safe over a wide range of parameters and allows high rates of reliable analog or digital communications. A small component that is typically driven in an untethered way is integrated with a mechanism that can register electric voltage between at least two points and transmit the resulting recorded signal. A repositionable, small component allows for versatile and safe monitoring of different intracranial sites. A wireless reliable communication method allows reconstructing high-fidelity signals and thus understanding brain activity in places that are otherwise inaccessible.

In a more specific context, without limiting the scope of the invention, in one embodiment, the above described mechanism is integrated with a magnetically actuated micro-robotic mechanism to traverse between different areas in the CNS, and thus allow repositionable monitoring of neural electrical activity in medical contexts such as epilepsy foci mapping and seizure recognition; movement disorders such as Parkinson's disease, essential tremor or dystonia; pain management; psychiatric disorders or many others.

In one of the preferred embodiments described herein, the system includes an optical transmitter such as a LED light, through which light is modulated to ‘flicker’ and transmit information. While the human brain is fairly opaque in most of the relevant electromagnetic spectrum, the cerebrospinal fluid (CSF) is highly transparent in some of it and can naturally scatter sufficient light to be detected for signal reconstruction. In addition, added materials injected to the CSF can induce and augment scattering. In yet another one of the described embodiments, an analog electric circuit on the intracranial implant component is used to modulate the neural signal on a carrying signal of a higher frequency, by, for example, amplitude modulation. The electric signal is transmitted through the human body and attenuated significantly but is detected via frequency filtering.

The invention herein shows a system with multiple embodiments to achieve all of the above described while maintaining the ability to integrate said mechanisms on a sufficiently small implant, and ideally a maneuverable intracranial component to the ability to reposition and monitor different intricate areas in the nervous system.

### BRIEF DESCRIPTION OF THE FIGURES

FIG. 1: A schematic representation of the main components of an embodiment based on optical modulation of data.

FIG. 2: A schematic representation of the main components of an embodiment based on galvanic human body communications (HBC).

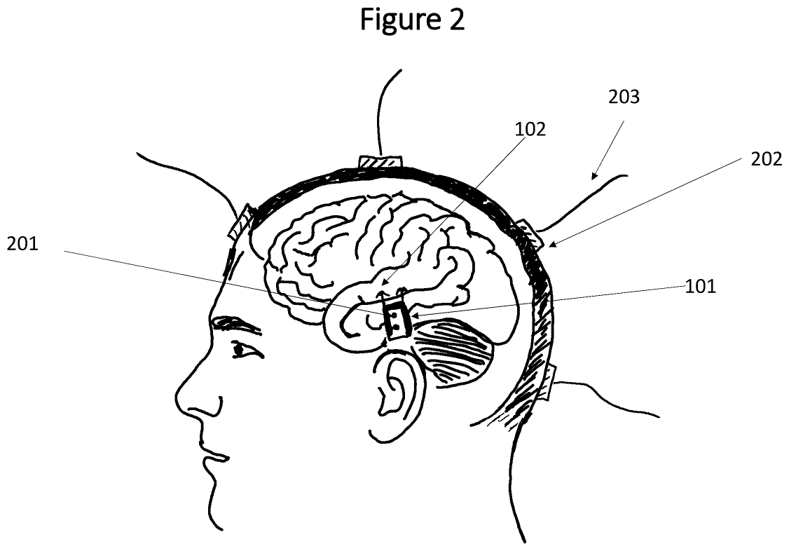

FIG. 3: A block diagram of the major components of one embodiment.

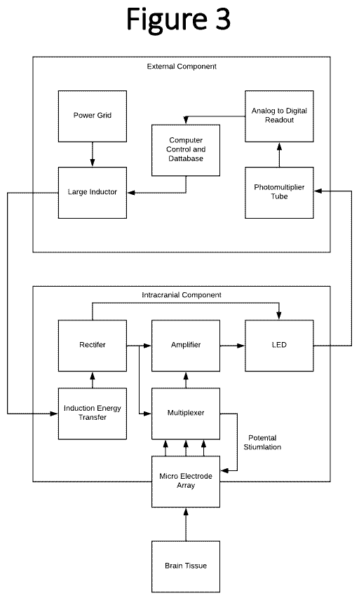

FIG. 4: A schematic representation of an electrocorticography (ECoG) array with a magnetic actuation mechanism for deployment and an optical signal transmission mechanism.

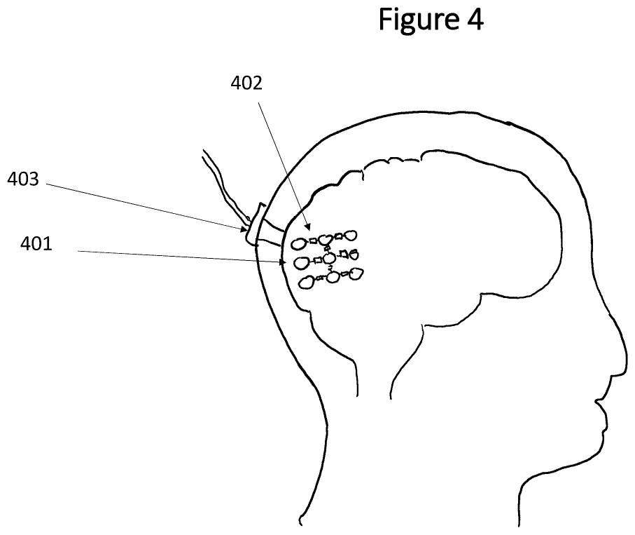

### DETAILED DESCRIPTION OF THE INVENTION

In reference to the figures, where like-numbers refer to same components, the invention described herein is a system for the purpose of monitoring neural activity in patients, and especially in clinical (human) patients suffering from variety of neurological disorders or diseases. Monitoring of neural activity can be, and is typically performed by electroencephalogram (EEG), meaning recording the electric fields or voltages between at least two points in or in close proximity to the nervous system. EEG has significant advantages in terms of cost, reliability and ease of use when compared to all other available tools. However, EEG is most commonly performed by placing electrodes on a patient's scalp, and thus only provides limited statistical information, representing an ensemble measurement over large and complex volumes, affected by signal attenuation and dispersion. In some medical cases, in order to acquire better resolution and signal quality, internal electrodes are placed intracranially. Electrodes can be placed in an array subdurally for electrocorticography (ECoG), or otherwise inside the brain tissue (e.g. stereo-EEG electrodes). In both cases, internal EEG is an invasive tool that requires significant recovery times and is limited by the brain anatomy and geometry to avoid health risks.

The invention herein serves to eliminate the limitations of existing EEG systems and extend on the aforementioned art to allow minimally invasive, versatile, maneuverable wireless internal EEG measurement, and specifically to enable the integration of local EEG sensing on micro-robotic platforms.

In order to achieve these goals, the invention includes an internal, meaning intracranial, component, which in the preferred embodiment is a magnetically actuated micro-robot (101), moved through either the CSF typically by exerting magnetic fields and magnetic fields'gradients exerting torques and forces on it to propel it through the cerebrospinal fluid (CSF) or through brain tissue, typically by applying torques and utilizing some mechanical leverage as a screw shape. The internal unit typically has at least 2 electrodes (102) intended for sensing the electric voltage between them. The data as is defined by the voltage signal between the two electrodes is typically amplified using an operational amplifier and is either converted to a digital format or used in its analog format. The signal then needs to be transmitted and recorded externally. When juxtaposed with the prior art described above, the invention herein circumvents the major problems of attenuation, potential harm, power limitations or volume limitations by using different mechanisms.

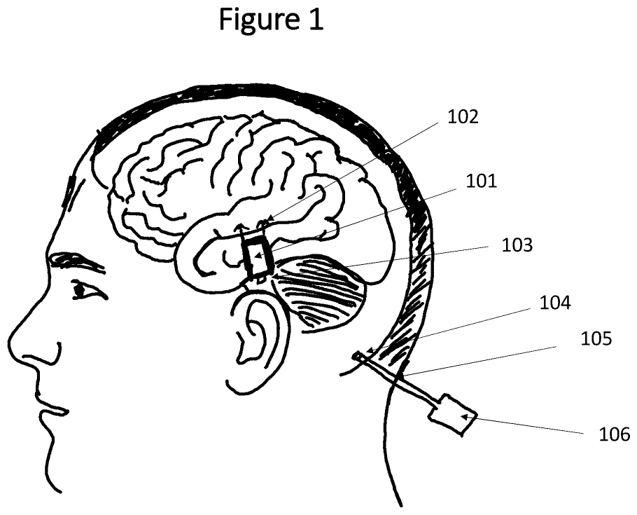

With reference to FIG. 1, the electrodes (102) are typically made of conductive materials intended to be stable over a range of temperatures and electrochemically stable as well as biocompatible. One of the electrodes can be referred to as a reference or a virtual ground, but typically both electrodes are made of the same material such as Pt, PtIr, Ag/AgCl, or another chemically stable material that does not tend to corrode in a saline environment.

In one embodiment, the transmission is based on a light signal such as an optical, infrared or other signal propagating through the brain tissue. The CSF is highly transparent to some parts of the spectrum, and specifically some parts of the optical spectrum. Since some natural reflections from the brain tissue and interfaces occur, as well as scattering an optical signal in the medium, some of the signal emitted from a light source can be detected away.

In one configuration, the system herein includes a light intensity detector such as photomultiplier tube placed through the same entry point used for injecting the device (101). Such entry point (105) can be placed for example as access to the cisterna magna, a burr hole in the occipital part, towards the frontal lobes, intranasal or intraocular as well as other locations. Through the entry point, a light guide can be introduced with some interface (104) to collect the scattered and reflected signal. This can be an optical fiber with or without a lens, a collimator, a filter, a polarizer and other devices. The light can then be guided and digitized through a photo multiplier tube (PMT) (106). The digital output is recorded and analyzed to reconstruct the assessed signa between the electrodes.

The light source (103) can be connected to a wireless power source such as an inductive power receiver through a rectifier or directly, and the voltage dictating the light intensity can be modulated via an amplifier to reflect the measured voltage between the electrodes. This would generate an amplitude modulated signal that can be read and demodulated through the reading component (106). Alternatively, other modulation options can be utilized. The light source can be kept in the CSF to avoid attenuation in the tissue. The light source can be a LED, laser diode, monochromatic, with several colors, with a filter or any combination thereof.

To augment the scattering of the light emitted from the source (103), modification can be made to the internal device (101), to the light source (103) itself. In some embodiments, some manipulation can be performed to increase the scattering of light through the media by, for example, introducing a particulate matter into the CSF. Such particulate matter, at size scales comparable to that of the emitted wavelengths or smaller than them, can scatter the light to enhance the intensity detected in the reading component (106). As another example, suspended particulates such as liposomes, micelles, nanoparticles, microbubbles, nanobubbles or other materials with or without chemical modifications can be introduced to the CSF in either a way that is biodegradable, biocompatible or a material that can be washed out of the intrathecal space.

In some embodiments, multiple wavelengths can be used simultaneously for multiple signal measurements. For example, several LEDs can be implemented as a light source (103). Alternatively, chromatic filters can be implemented and exchanged, or a single multi-wavelengths source can be used. In a similar fashion, information can be coded based on polarization, phase, pulse width or other known method.

In another embodiment, the reading component (106) can be placed internally, in an intracranial place, in the cisterna magna, or in any other place in the brain, to serve as a readout point and retransmit the information. Multiple sensors can be placed in different locations, or multiple sensors with light transmitting components on each to retransmit the data in a distributed way and validate sufficient light intensity reaches the external components. In some cases, the system can be configured to have photodiodes for both light emission and light intensity reading to save volume, or to use the diode inherent non-linearity as part of the modulation mechanism. The device can further use a memory component to transmit at a different time than the receiving.

With reference to FIG. 1, the system can further use electrical current communications, typically referred to as human-body-communications (HBC). HBC is advantageous over the prior art in emitting encephalography signals from the brain in that it allows extensive filtering. For example, a signal between two internal electrodes (102) can be amplified and modulated over a carrier frequency such as 10 KHz or 100 KHz. The resulting high frequency signal with modulated amplitude can be safely induced as a current between two other electrodes (201). The resulting current primarily flows between said electrodes but can be detected at low levels via notch filtering for the frequency. With two adjacent electrodes for the transmission being at a distance of several millimeters apart, the expected resistance between them is 3-4 orders of magnitude lower than that to an external electrode that is located typically several centimeters away through tissue.

An example of the amplitude modulation can be performed through a single diode as a nonlinear component, with the higher frequency generated in an oscillator on board the internal unit, or via a mutual induction in a coil on the internal unit and an AC supply externally.

The detection of the residual current externally can be done via means of lock-in amplifiers, bandpass filters, low-noise amplifiers, chopper amplifiers, cryogenic amplifiers and others, wherein signal processing can be applied in an analog or a digital form.

The electrodes (201) can be manipulated mechanically to be farther away from each other by a magneto-mechanical component, a shape memory component, an electromechanical actuator or other means to increase the resistance between the electrodes and improve the signal. The detection of the output signal can be performed with 2 or more electrodes (202) that are typically placed extra-cranially, or intra-cranially in close proximity to the skull, by setting them through a burr hole or another access point. The electrodes are then galvanically connected (203) to aforementioned amplification and filtering components.

In some embodiments, the signal transmission is performed magnetically, where the signal of interest is modulated onto some motion of a permanent magnet component or a current flowing in an internal coil. The permanent magnet moving can be the same one used for mechanical actuation in the preferred embodiment, a smaller magnet, a magnet on a cantilever, two magnets vibrating one in reference to the other or another configuration. The magnetic field changes are typically detected via a high sensitivity magnetic sensor placed extracranially, or intracranially in close proximity to the skull, such as SQUIDs, fluxgate sensors, giant magneto resistive sensors, tunneling magneto resistive sensors, hall sensors or others.

All of the embodiments above refer to the measurement of an electric voltage as defined by two or more electrodes. However, other measures or different sensors can be implemented in conjunction with said electrodes, in addition or separately without them. For example, ion-specific field effect transistors (ISFETs) can be configured to detect such materials as specific metal ion concentrations, lactate, ascorbic acid, or others. Functionalized membranes can be implemented to detect specific neurotransmitters such as dopamine or serotonin. Sensors for magnetic or electric field can be implemented on the internal unit in addition or instead of the voltage measurement. In some embodiments additional environment sensors can be used such as pressure or temperature, for data transmission or for corrections and signal processing of the acquired signal. In a different embodiment, the components mentioned above for sensing and transmitting an EEG signal wirelessly are implemented in an array of sensors that is implanted intracranially and without piercing through the parenchymal tissue. This configuration, when juxtaposed with ECoG arrays as were shown in the art and applied clinically, can be implanted in a minimally invasive manner that does not require craniotomy because of the wires volume that is not required. In addition, in some embodiments, an array of electrodes (401) can be configured to be deployed magnetically, by, for example, integrating a permanent magnet on each electrode, integrating a permanent magnet and geometry that allows motion through narrow liquid spaces such as a screw shape, a corkscrew shape, a buckling component, a wobbling component, a sliding component or other. In some embodiments, the electrodes are inter-connected all with wires, joints or connectors, and some electronics that include a light emitting component (402).

The electronics between electrodes can transmit data modulated with aforementioned techniques to be received via a light measuring device (403) such as a PMT as described above.

The invention described is applicable to a wide variety of CNS and PNS related diseases and conditions, such as and not limited to—epilepsy procedures for detection of epileptic zones in a patient brain for diagnosis and towards treatment; monitoring of the focal points and progression of movement disorders such as Parkinson's disease, dystonia, essential tremor, Huntington's disease or ALS, diagnosis and treatment of pain disorders and aid in pain management solutions including via measuring and stimulating the brainstem, the thalami and other parts; Diagnosis and treatment of psychiatric disorders; Diagnosis and treatment of tinnitus; Diagnosis and treatment of insomnia and many others.

The invention can further serve as a component in a system to provide nerve stimulation for a variety of medical indications, such as Vagus nerve stimulation with a close loop mechanism that detects EEG signals, transmits and potentially analyzes the data and accordingly stimulates the Vagus nerve. Such a closed loop can be used to augment existing nerve stimulation devices, to improve response over time, to utilize biofeedback and user feedback to the stimulation result or otherwise serve as a medical solution. The stimulation can be done via external electrodes, internal electrodes, the electrodes on the internal unit (102) or any of the plurality of combinations thereof.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed:

1. A system for monitoring, registering or recording neural activity as defined by the electric voltage between at least two electrodes, which includes an intracranial wireless component and an extracranial component, the system comprising:
a. at least two electrodes, of which one or more can be a reference electrode, in the
intracranial device;
b. a means for modulating the analog voltage signal onto a transmission mechanism in the intracranial device;
c. a wireless transmission mechanism in the intracranial device; and
d. a means for receiving the transmitted signal extracranially or close to the cranium.

2. The system of claim 1 where the transmission mechanism is based on human-body-conduction, meaning an electrical current is generated at a higher frequency than the signal and read extracranially.

3. The system of claim 1 where the transmission mechanism is based on light emission at wavelengths such as the optical spectrum or parts of the near infrared spectrum, emitted intracranially and scattering thereof measured via a transcranial such as through an orifice or via an intracranial component located proximally.

4. The system of claim 3 where the light scattering is enhanced by manipulating the cerebrospinal fluid.

5. The system of claim 4 where scattering is enhanced by introducing a biocompatible particulate matter such as biodegradable polymers or liposome-based agents.

6. The system of claim 1 where the receiving mechanism is a photomultiplier tube (PMT).

7. The system of claim 1 where the receiving mechanism allows discerning between several wavelengths, for example from multiple internal units.

8. The system of claim 1 where the receiving mechanism protrudes through the skull, for example through a burr hole.

9. The system of claim 1 where the receiving mechanism is implanted in a fluid volume close to the surface of the skull and communicates via other means externally.

10. A system for electrocorticography (ECoG) with an array of at least 4 electrodes and at least 4 transmitting elements, where the transmitting elements are configured to transmit the differential signal between some or all of the electrode pairs.

11. The system of claim 10 where the electrodes array is spatially manipulated by an external magnetic field or gradient.

12. The system of claim 10 where the electrodes are connected via magnetically controlled joints.
